(19) United States
(12) Patent Application Publication
(10) Pub. No.: US 2024/0189075 A1
(43) Pub. Date: Jun. 13, 2024

(54) BONE FIXTURE WITH VARIOUS MODULI

(71) Applicant: INGROWTH BIOTECH CO., LTD., Kaohsiung City (TW)

(72) Inventors: Jason SUN, Kaohsiung City (TW); Royal Su, Kaohsiung City (TW); Po-Han Chu, Kaohsiung City (TW); Liang-Chueh Chung, Kaohsiung City (TW); Jenny Zwei-Chieng Chang, Kaohsiung City (TW)

(21) Appl. No.: 18/150,815

(22) Filed: Jan. 6, 2023

(30) Foreign Application Priority Data
Dec. 12, 2022 (TW) 111147616

(51) Int. Cl. A61C 8/00; A61K 6/84

(52) U.S. Cl. CPC A61C 8/0037; A61K 6/84

(57) ABSTRACT

A bone fixture with various moduli has an implant body. The implant body includes a first implanting segment adapted to be implanted into a cortical bone of a bone and a second implanting segment adapted to be implanted into a cancellous bone of the bone and located below the first implant segment. A first young's modulus of the first implanting segment is smaller than a second young's modulus of the second implanting segment. Because the first young's modulus of the first implanting segment adapted to be implanted in the cortical bone is smaller than the second young's modulus of the second implanting segment adapted to be implanted in the cancellous bone, forces applied to the bone fixture can be evenly distributed to avoid the stress concentration at the cortical bone of the bone.

### BACKGROUND OF THE INVENTION

### 1. Field of the Invention

The present invention relates to a human body implant, and more particularly to a bone fixture configured to be implanted into a bone.

### 2. Description of Related Art

A bone fixture, such as a bone screw or an implant, is mainly adapted to be implanted into a bone in combination with the osseous tissues. The bone has a cortical bone located at an outer layer of the bone and a cancellous bone located inside the cortical bone. The cortical bone is hard and dense. The cancellous bone has a density lower than that of the cortical bone and is flexible.

With reference to FIG. 8, a conventional implant 90 is mainly made of metal and is solid. When the implant 90 is implanted into an alveolar bone 80, the implant 90 is inserted through the cortical bone 81 at the outer layer of the alveolar bone 80 and extends into the cancellous bone 82 of the alveolar bone 80. After the alveolar bone 80 is osseointegrated with the implant 90, an abutment and a crown are connected to the implant 90.

The implant 90 is adapted to support the crown for bearing bite force. When the implant 90 is loaded, as a stress distribution region 70 shown in FIG. 8, the cortical bone 81 of the alveolar bone 80 contacts an upper segment 91 of the implant 90 and bears a larger stress than the cancellous bone 82 of the alveolar bone 80. The cancellous bone 82 contacting a lower segment 92 of the implant 90 is almost free from stress. Accordingly, stress applied to the implant 90 cannot be evenly distributed. The bone density of the cortical bone 81 surrounding the upper segment 91 of the implant 90 is easily reduced because of stress concentration and overloading. Whereby, the stability of the implant 90 fixed in the alveolar bone 80 is decreased, and the implant 90 will easily detach from the alveolar bone 80 in the long term.

To overcome the shortcomings of the conventional implant, the present invention tends to provide a bone fixture with various moduli to mitigate or obviate the aforementioned problems.

### SUMMARY OF THE INVENTION

The main objective of the present invention is to provide a bone fixture with various young's moduli to evenly distribute stress and to mitigate the problem of stress concentration at the cortical bone.

The bone fixture with various moduli in accordance with the present invention is adapted to be implanted into a bone and comprises an implant body. The implant body includes a first implanting segment adapted to be implanted into a cortical bone of the bone and having a first young's modulus and a second implanting segment adapted to be implanted into a cancellous bone of the bone, located below the first implanting segment, and having a second young's modulus. Wherein the first young's modulus of the first implanting segment is smaller than the second young's modulus of the second implanting segment.

Other objectives, advantages and novel features of the invention will become more apparent from the following detailed description when taken in conjunction with the accompanying drawings.

### BRIEF DESCRIPTION OF THE DRAWINGS

FIG. 1 is a side view of a first embodiment of a bone fixture with various moduli in accordance with the present invention;

FIG. 2 is a cross sectional side view of the bone fixture in FIG. 1;

FIG. 3 is an operational cross sectional side view of the bone fixture in FIG. 1 showing that the bone fixture is implanted into a bone;

FIG. 4 is a cross sectional side view of a second embodiment of a bone fixture in accordance with the present invention;

FIG. 5 is a cross sectional side view of a third embodiment of a bone fixture in accordance with the present invention;

FIG. 6 is a schematic side view of the bone fixture implanted into a bone in FIG. 1, showing a stress distribution region in a bone implanted by the bone fixture when the bone fixture is pressed by a vertical force;

FIG. 7A is a diagram of a finite element analysis simulation, showing a stress distribution inside a cancellous bone of a bone which is implanted by the bone fixture in accordance with the present invention when the bone fixture is subjected to an oblique force;

FIG. 7B is a diagram of a finite element analysis simulation, showing a stress distribution inside a cancellous bone of a bone which is implanted by a conventional implant when the conventional implant is subjected to an oblique force;

FIG. 7C is a diagram of a finite element analysis simulation, showing a stress distribution inside a cancellous bone of a bone implanted by a bone fixture, wherein the bon fixture, subjected to an oblique force, has a first implanting segment and a second implanting segment with a young's modulus smaller than a young's modulus of the first implanting segment; and

FIG. 8 is a schematic side view of a conventional implant implanted into an alveolar bone, showing a stress distribution region in the alveolar bone when the conventional implant is pressed by a vertical force.

### DETAILED DESCRIPTION OF THE PREFERRED EMBODIMENT

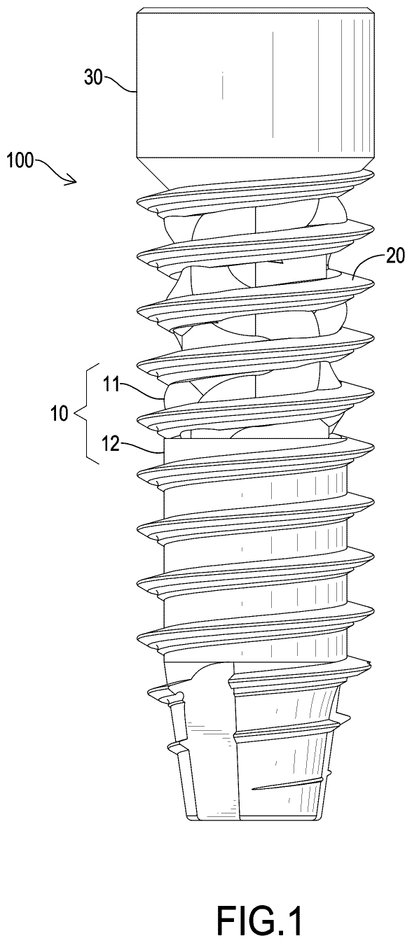

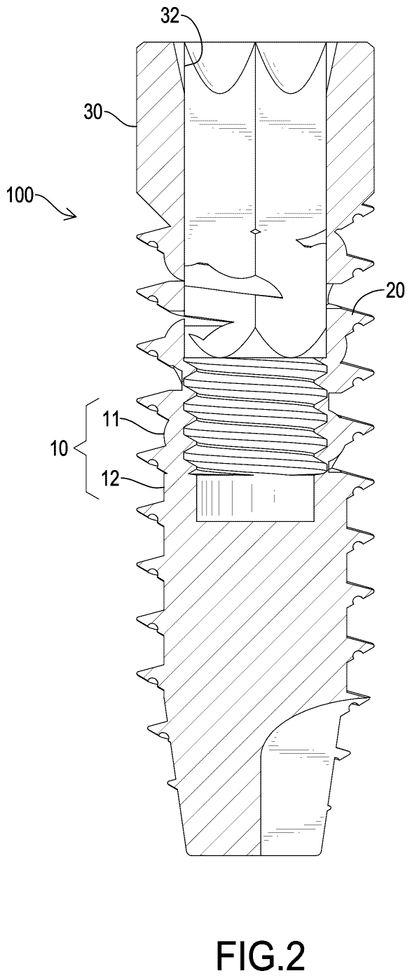

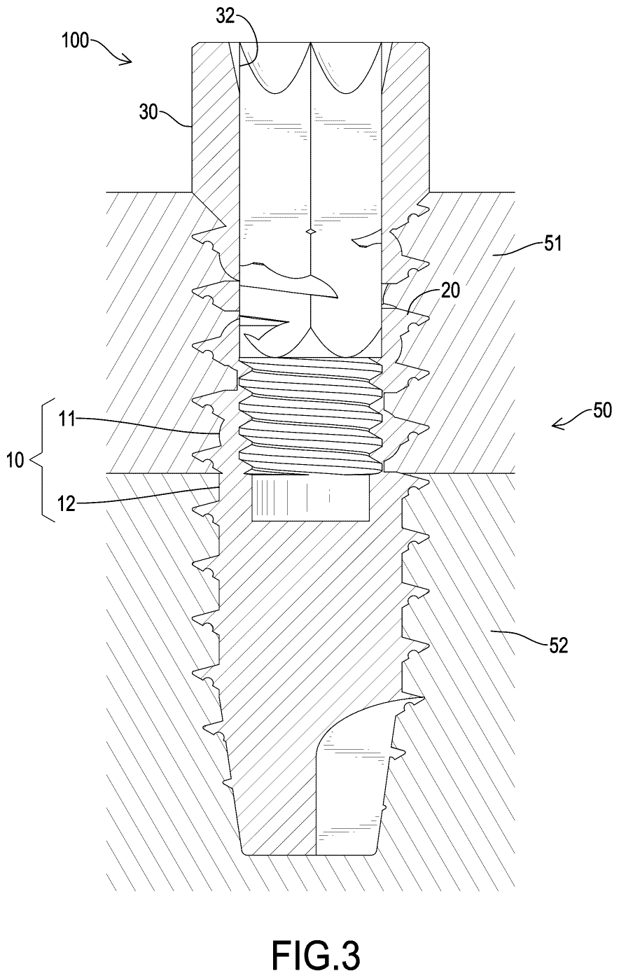

With reference to FIGS. 1 to 3, a first embodiment of a bone fixture 100 with various moduli in accordance with the present invention is adapted to be implanted into a bone 50. The bone 50 includes a cortical bone 51 located at an outer layer of the bone 50 and a cancellous bone 52 located inner than the cortical bone 51. The bone fixture 100 comprises an implant body 10. The implant body 10 includes a first implanting segment 11 and a second implanting segment 12. The first implanting segment 11 is adapted to be implanted into the cortical bone 51 of the bone 50 and has a first young's modulus. The second implanting segment 12 is adapted to be implanted into the cancellous bone 52 of the bone 50, is located below the first implanting segment 11, and has a second young's modulus. The first young's modulus of the first implanting segment 11 is smaller than the second young's modulus of the second implanting segment 12. Preferably, the first young's modulus of the first implanting segment 11 ranges from 0.01 GPa to 40 GPa, and more preferably the first young's modulus ranges from 0.01 GPa to 14 GPa. The second young' modulus of the second implanting segment 12 may be 80 GPa.

In the first embodiment, the second implanting segment 12 is solid, e.g. a solid metal material. The first implanting segment 11 may have a porous structure, e.g. a porous metal material. The first implanting segment 11 and the second implanting segment 12 may be made of a same metal, e.g. titanium, titanium alloy, and tantalum alloy. Because of the first implanting segment 11 being porous, the first young's modulus of the first implanting segment 11 is smaller than the second young's modulus of the second implanting segment 12. The bone fixture 100 with various moduli may be manufactured by 3D printing technology to form the first implanting segment 11 being porous and the second implanting segment 12 being solid. The first implanting segment 11 is porous, allowing osteoblasts of the cortical bone 51 to proliferate therein.

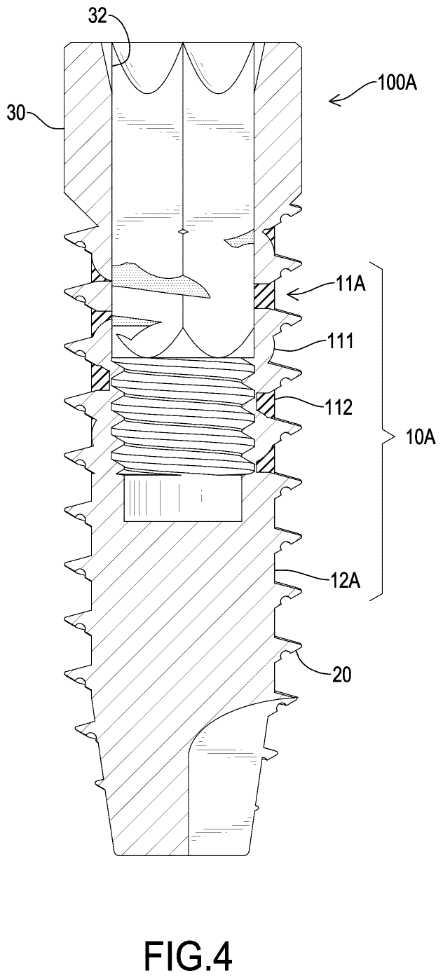

With reference to FIG. 4, in the second embodiment of the bone fixture 100A in accordance with the present invention, the second implanting segment 12A of the implant body 10A is solid, and the first implanting segment 11A of the implant body 10A includes a porous body 111 and a filler 112. The porous body 111 has a porous structure and the first implanting segment 11A and the second implanting segment 12A may be made of a same material. The filler 112 is made of a polymer filling material and is filled into the porous structure of the porous body 111. With such structure, the first young's modulus of the first implanting segment 11A is still smaller than the second young's modulus of the second implanting segment 12A. The filler 112 filled in the porous body 111 enhances a structural strength of the first implanting segment 11A.

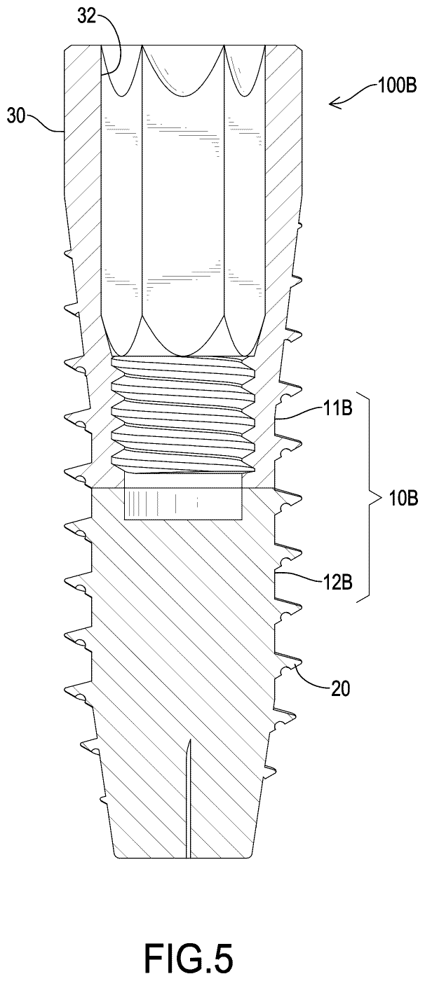

With reference to FIG. 5, in the third embodiment of the bone fixture 100B in accordance with the present invention, the first implanting segment 11B and the second implanting segment 12B of the implant body 10B are both solid. The first implanting segment 11B and the second implanting segment 12B are made of different materials. Wherein, the first implanting segment 11B may be made of solid tantalum alloy, and the second implanting segment 12B may be made of solid titanium alloy. With different materials, the first young's modulus of the first implanting segment 11B is smaller than the second young's modulus of the second implanting segment 12B.

With reference to FIGS. 1 to 3, the bone fixture 100 with various moduli further comprises a thread 20, a mounting portion 30, and a connecting cavity 32. The thread 20 spirally surrounds the implant body 10. The mounting portion 30 is formed at a top of the implant body 10 and is adapted to support an abutment. The connecting cavity 32 is formed at a top of the mounting portion 30, extends downwardly from the top of the mounting portion 30, and extends into the implant body 10. The mounting portion 30 is adapted to connect with the abutment. An inner thread may be spirally formed within the connecting cavity 32.

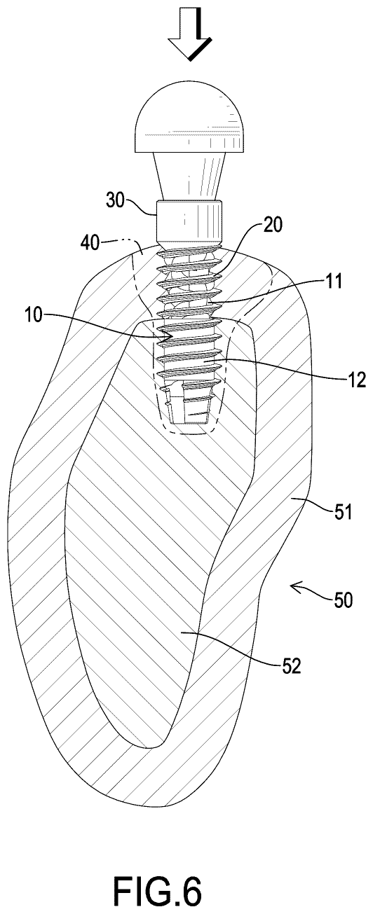

With reference to FIG. 6, the bone fixture 100 is implanted into the bone 50 and a vertical downward force is applied to the bone fixture 100. As a stress distribution region 40 shown in FIG. 6, the cortical bone 51 surrounding the first implanting segment 11 of the implant body 10 and the cancellous bone 52 surrounding the second implanting segment 12 of the implant body 10 both receive the stress. Accordingly, the force applied to the bone fixture 100 can be distributed to the cancellous bone 52 to avoid the stress concentration at the cortical bone 51.

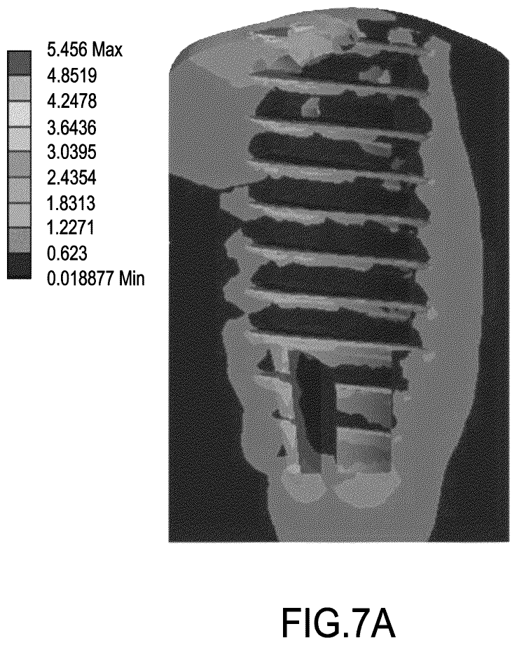

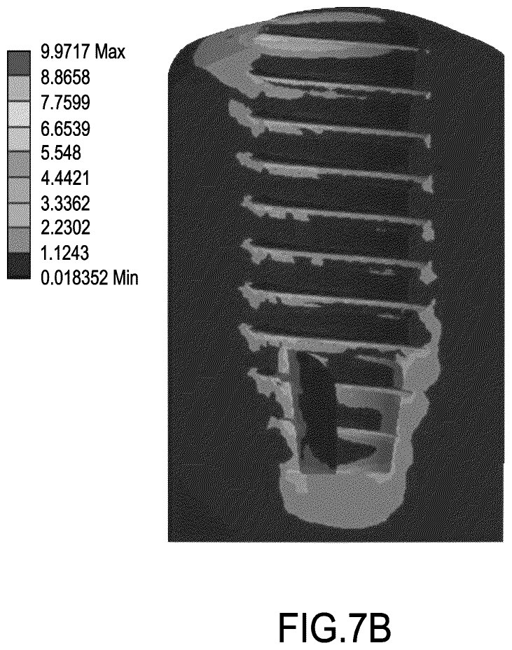

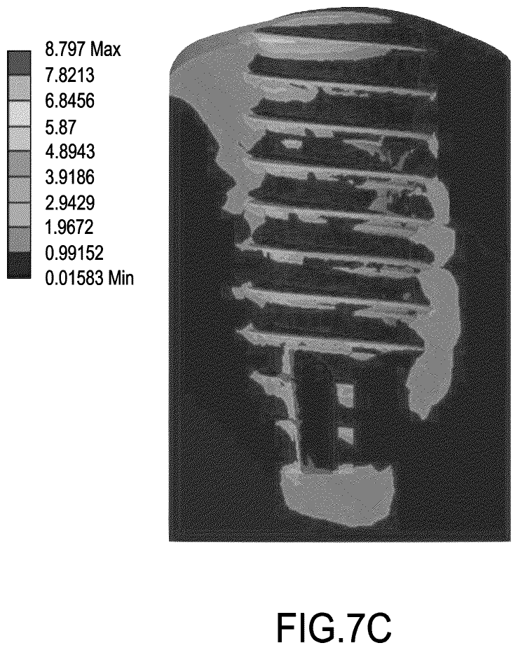

FIG. 7A is a diagram of a finite element analysis simulation showing that a stress distribution inside the cancellous bone of the bone which is implanted by the bone fixture in accordance with the present invention, subjected by an oblique fore, in accordance with the present invention. FIG. 7B is a diagram of a finite element analysis simulation showing that a stress distribution inside a cancellous bone of a bone which is implanted by a conventional implant subjected by the same oblique force. FIG. 7C is a diagram of a finite element analysis simulation showing that a stress distribution inside a cancellous bone of a bone which implanted by a bone fixture different from the bone fixture 100 of the present invention, wherein the bon fixture has a first implanting segment and a second implanting segment with a young's modulus smaller than a young's modulus of the first segment.

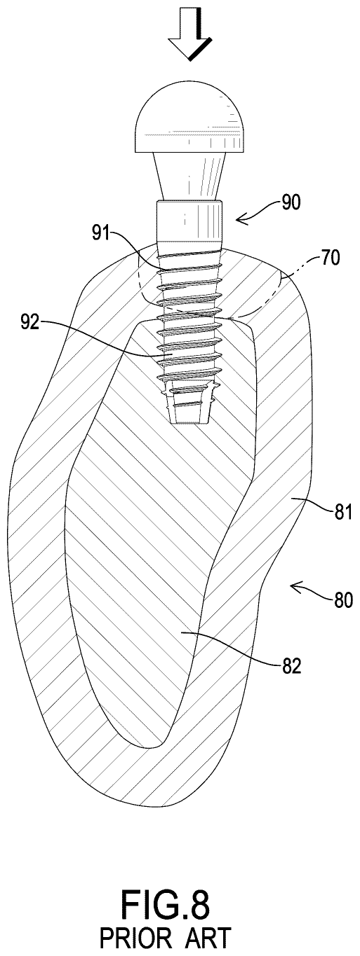

With reference to FIGS. 6 and 7A, the bone fixture 100 in accordance with the present invention is implanted into the bone 50 and an oblique force (a resultant force of a vertical downward force being 150 newton (N) and a lateral force being 20 newton (N)) is applied to the bone fixture 100. The maximum stress detected at the first implanting segment 11 of the bone fixture 100 implanted into the cortical bone 51 is 35.885 MPa, and the maximum stress detected at the second implanting segment 12 implanted into the cancellous bone 82 is 5.456 MPa. As shown in FIG. 7A, the stress is evenly distributed around the whole bone fixture 100. With reference to FIGS. 7B and 8, when a same oblique force is applied to a conventional implant 90 as shown in the FIG. 8, the maximum stress detected at the upper segment 91 of the conventional implant 90 implanted into the cortical bone 81 is 82.604 MPa, and the maximum stress detected at the lower segment 92 of the conventional implant 90 implanted into the cancellous bone 82 is 9.9717 MPa. With reference to FIG. 7B, the maximum stress is detected at a threaded, disposed between the cortical bone 81 and the cancellous bone 82, of the conventional implant 90. As shown in FIG. 7B, the stress is unevenly distributed at a side with respect to the conventional implant 90. With reference to FIG. 7C, when the same oblique force is applied to a bone fixture having the first implanting segment and the second implanting segment with a young's modulus smaller than an young's modulus of the first implanting segment, the maximum stress detected at the first implanting segment of the bone fixture inside the cortical bone is 75.215 MPa, and the maximum stress detected at the second implanting segment of the bone fixture implanted into the cancellous bone is 8.797 MPa. As shown in FIG. 7C, the stress is unevenly distributed at a side with respect to the bone fixture.

In addition, when the same vertical downward force or the same oblique force is applied to the bone fixture 100 in accordance with the present invention and the conventional implant 90, the maximum stresses detected at the cortical bone 51 implanted by the bone fixture 100 of the present invention are lower than the maximum stresses detected at cortical bone 81 implanted by the conventional implant 90.

According to the above-mentioned experiments, it can be assumed that because the first young's modulus of the first implanting segment 11 is smaller than the second young's modulus of the second implanting segment 12, the stress applied to the first implanting segment 11 can be evenly distributed. Accordingly, the stress detected at the first implanting segment 11 of the bone fixture 100 implanted in the cortical bone 51 is smaller than the stress detected at the upper segment 91 of the conventional implant 90 implanted in the cortical bone 81. Moreover, because the stress applied to the second implanting segment 12 can be evenly distributed into the cancellous bone 52, the stress detected at the second implanting segment 12 of the bone fixture 100 implanted in the cancellous bone 52 is smaller than the stress detected at the lower segment 92 of the conventional implant 90 implanted in the cancellous bone 82.

Accordingly, the bone fixture 100 of the present invention provides a preferable stress distribution. Compared with the conventional implant 90, the bone fixture 100 of the present invention is subjected to a lower stress. A force applied to the bone fixture 100 can be distributed evenly to avoid the stress concentration in the cortical bone 51, thereby enhancing the stability of the combination of the bone fixture 100 and the bone 50.

What is claimed is:

1. A bone fixture with various moduli adapted to be implanted into a bone and comprising:
an implant body including
a first implanting segment adapted to be implanted into a cortical bone of the bone and having a first young's modulus; and
a second implanting segment adapted to be implanted into a cancellous bone of the bone, located below the first implanting segment, and having a second young's modulus; wherein the first young's modulus of the first implanting segment is smaller than the second young's modulus of the second implanting segment.

2. The bone fixture with various moduli as claimed in claim 1, wherein the first young's modulus of the first implanting segment of the implant body is 0.01 GPa to 40 GPa.

3. The bone fixture with various moduli as claimed in claim 1, wherein the first young's modulus of the first implanting segment of the implant body is 0.01 GPa to 14 GPa.

4. The bone fixture with various moduli as claimed in claim 2, wherein
the second implanting segment of the implant body is solid; and
the first implanting segment of the implant body is porous.

5. The bone fixture with various moduli as claimed in claim 2, wherein
the second implanting segment of the implant body is solid; and
the first implanting segment of the implant body has a porous body and a filler; the porous body has a porous structure; and the filler is made of a polymer filling material and filled into the porous structures of the porous body.

6. The bone fixture with various moduli as claimed in claim 2, wherein the first implanting segment is solid and made of a tantalum alloy.

7. The bone fixture with various moduli as claimed in claim 1, wherein the bone fixture with various moduli comprises a thread spirally surrounding the implant body.

8. The bone fixture with various moduli as claimed in claim 2, wherein the bone fixture with various modulus comprises a thread spirally surrounding the implant body.

9. The bone fixture with various moduli as claimed in claim 3, wherein the bone fixture with various moduli comprises a thread spirally surrounding the implant body.

10. The bone fixture with various moduli as claimed in claim 4, wherein the bone fixture with various moduli comprises a thread spirally surrounding the implant body.

11. The bone fixture with various moduli as claimed in claim 5, wherein the bone fixture with various moduli comprises a thread spirally surrounding the implant body.

12. The bone fixture with various moduli as claimed in claim 6, wherein the bone fixture with various moduli comprises a thread spirally surrounding the implant body.
